(19) United States
(12) Patent Application Publication
(10) Pub. No.: US 2017/0233703 A1
(43) Pub. Date: Aug. 17, 2017

(54) Genetic indicator and control system and method utilizing split Cas9/CRISPR domains for transcriptional control in eukaryotic cell lines

(71) Applicant: Tsinghua University, Beijing (CN)

(72) Inventors: Zhen Xie, Beijiing (CN); Shuguang Peng, Beijing (CN); Dacheng Ma, Beijing (CN)

(73) Assignee: Tsinghua University, Beijing (CN)

(21) Appl. No.: 15/376,569

(22) Filed: Dec. 12, 2016

(30) Foreign Application Priority Data
May 21, 2015 (CN) 20151263106
May 20, 2016 (CN) 201610341363.0

(51) Int. Cl. C12N 9/22; C12N 15/86; A61K 48/00; C12N 7/00; C12N 15/82

(52) U.S. Cl. CPC C12N 9/22; C12N 7/00; C12N 15/8213; A61K 48/0075; A61K 48/005; C12N 15/86; C12N 2710/10343

(57) ABSTRACT

While genetic engineering has undergone rapid advancement with the discovery of CRISPR/Cas9, there is room for improvement for genetic circuit control, precision (reducing circuit ‘leakiness’) and delivery into living systems. The claimed invention offers programmable and precise regulation of dCas9 functions in response to multiple molecular signals by using synthetic gene circuits, greatly expanding applications. Moreover, using the system to greatest therapeutic potential has been greatly limited by the restrictive cargo size of existing viral delivery systems. By splitting dCas9 into multiple sections, the delivery size of synthetic gene circuits is greatly reduced. By exchanging split dCas9 domains, differential regulation on one gene, or activating two different genes in response to cell-type specific microRNAs is illustrated. Practical applications of the illustrative examples include engineered sensory switches including indicators for bladder cancer as well as enhanced systems for adenovirus delivery, cellular regulation, plant cell modification and potential therapeutic applications.

### SEQUENCE LISTING

The instant application contains a Sequence Listing which has been submitted in ASCII format via EFS-Web and is hereby incorporated by reference in its entirety. Said ASCII copy, created on Nov. 13, 2016, is named ZX1seqlist_ST25.txt and is 321 kbytes in size.

### RELATED APPLICATIONS

This application claims priority to China Application number 201610341363.0 filed May 20, 2016 which is a continuation of China Application CN20151263106 2015052 filed May 21, 2015.

### BACKGROUND OF THE INVENTION

The CRISPR-associated protein 9 (Cas9) discovered from Streptococcus pyogenes is a multi-domain protein, which has been widely used in genome editing and transcriptional control in mammalian cells due to its superior modularity and versatility. Delivering synthetic gene circuits in vivo has been limited due to size constraints particularly with smaller delivery systems with a payload capacity nearly equal to an entire Cas9 complex.

### SUMMARY OF THE INVENTION

Several strategies have been developed to engineer modular and layered gene circuits in mammalian cells by regulating dCas9 and gRNA expression. Transcriptional controls in mammalian cells can be achieved by directly fusing a transcriptional regulatory domain to the nuclease deactivated Cas9 (dCas9). Alternatively, multiple transcriptional regulatory domains can be recruited to the dCas9 by tagging the dCas9 with a repeating peptide scaffold, or by fusing repeating RNA motifs to the cognate gRNA. However, biomedical applications of the CRISPR/Cas system require the exploration of new platforms for engineering mammalian synthetic circuits that integrate and process multiple endogenous inputs. In addition, the application of CRISPR/Cas therapeutic circuits is also challenging due to the restrictive cargo size of existing viral delivery vehicles.

The split Cas9 system can be used in general to bypass the packing limit of the viral delivery vehicles and in the claimed invention dCas9 is split and reconstituted in human cells. One of the challenges of therapeutic applications is to find an optimal delivery system that can carry all CRISPR/Cas9 components to the desired organ or cell population for genetic manipulation. Using the CRISPR/Cas system to greatest potential has been greatly limited by its physical size when incorporated into a viral delivery system. When used for synthetic biology purposes in high value delivery systems with site specific integration such as the Adeno-Associated Virus/AAV, the entire cas9 complex is akin to a computer operating system taking up 95% of available memory leaving only a small portion for synthetic biology programming purposes. By splitting the CRISPR/CAS9 into smaller regions and delivering the regions in separate viral delivery vectors, the powerful genetic manipulation functionality is retained alongside substantial increases in space for cellular programming purposes. The claimed invention represents a substantial improvement over existing CAS9 delivery techniques and includes additional enhancements for genetic control and programming.

While a variety of viral delivery systems have been employed with mixed success, implementation of systems relying on alternate virus systems can lead to an undesired strong immune response. Using the recombinant adeno-associated virus (rAAV) offers high gene transfer efficiency and very low immune response. Unfortunately packaging capacity is confined to 4.7 kb to 5 kb which is problematic when compared with human optimized Cas9 size at over 4.2 kb with promoter sequences reaching over 5 kb.

With intein-mediated split Cas9, inteins function as protein introns and are excised out of a sequence and join the remaining flaking regions (exteins) with a peptide bond without leaving a scar. In terms of split site selection particular attention is given to split sites which are surface exposed due to the sterical need for protein splicing. This system allows the coding sequence of Cas9 to be distributed on a dual-vector or multi-vector system and reconstituted post-translationally. In one illustrative embodiment, logic AND circuits and sensory switches are engineered and implemented by integrating and swapping split-dCas9 domains, which may reduce the size of synthetic circuits comparing to the circuits that use the full-length dCas9. Small molecule, shRNA or cell-type specific miRNA inputs are connected to control these Cas9-based synthetic circuits to enable new biomedical applications by using the CRISPR/Cas system.

The claimed invention expands the reach of synthetic biology by targeting specific diagnostic and therapeutic applications through improvements in genetic circuitry and higher level genetic circuit delivery enhancements. The claimed embodiments of the invention overcome existing size limitations through optimal splitting of Cas9 allowing for higher level synthetic gene circuitry to be accommodated by smaller delivery systems. In one embodiment of the invention, Cas9 is intein split at residues 203-204, 468-469, 713-714 and 1153-1154. In a complementary embodiment, split Cas9 fragments across different split pairs yield combinations that provided the complete polypeptide sequence activate gene expression even when fragments are partially redundant.

The structural analysis of the SpCas9:DNA:gRNA complex has facilitated the engineering of mutant SpCas9 proteins that recognize variant PAM sequences. For example, D1135E/R1335Q/T1337R mutations (EQR) or D1135V/G1218R/R1335E/T1337R mutations (VRER) in the PI domain can switch the PAM specificity of SpCas9 from NGG to NGCG. Furthermore, the functional Cas9 protein can be reconstituted from two inactive split-Cas9 peptides in the presence of gRNA by using a split-intein protein splicing strategy by respectively fusing to dipartite domains that interact with each other. Inteins often require cysteine, serine, or threonine at the +1 amino acid position immediately downstream of the C-terminal intein fragment to complete the self-catalytic splicing reaction. In the split-intein protein splicing system, the split Cas9 fragments are fused to either a N-terminal intein fragment or a C-terminal intein fragment, which can associate with each other and catalytically splice the two split Cas9 fragments into one Cas9 protein.

In an additional complementary embodiment, Cas9 fragments fused to regulatory domains such as Krab, VPR, Suntag and VP64 provide higher level control. Using the claimed invention, split dCas9 domains are reconstituted for transcriptional regulations in cultured human cells, allowing modular and efficient construction of three-input logic AND circuits in an illustrative embodiment. In an additional embodiment of the split dCas9 and Suntag system, it is possible to easily increase the number of inputs up to seven, including three split dCas9 domains, two Suntag fragments, the rapalog and the gRNA. In another embodiment of the claimed invention, by introducing mutations in the PI domains an orthogonal split dCas9 pair is disclosed which recognizes the NGCG PAM sequences instead of the NGG PAM sequences. The claimed orthogonal split dCas9 pairs are a useful toolkit to construct complex and layered logic gates with multiple inputs. In addition, foreseeable variants include utilizing a similar strategy to engineer split Cas9 pairs with nuclease or nickase activity.

Additional embodiments of the invention include the successful introduction of multiple input logic AND circuitry through the splitting dCAS9 into more than two fragments. Claimed enhancements include novel solutions to genetic circuit precision where genetic circuitry ‘leakiness’ is greatly reduced through the utilization of a feed forward loop enabling higher level circuit complexity.

In a further illustrative example of the claimed invention, application of the split-dCas9 system to bladder cancer cells is hereby demonstrated. By applying genetic circuitry AND gates to promoters hTERT (human telomerase reverse transcriptase) and hupII (human UroplakinII), specificity is demonstrated for genetic circuitry upon bladder cancer cells for diagnostic and potential therapeutic applications. In addition, increased specificity can be obtained through an inducible promoter such as TRE can be used to express gRNA in addition to Dox-induction.

The enhanced split CAS9 system with enhanced regulatory control has immediate relevance in biomedical applications, including but not limited to the claimed diagnostic detection system for bladder cancer as well as potential therapeutic applications. The claimed system will complement existing strategies to control the Cas9/dCas9 activity by using small molecules and light signals. Intended variants include connecting tissue and cellular specific inputs to regulate Cas9/dCas9 activity to facilitate the application of the CRISPR/Cas system in basic and translational biomedical research and biomedical applications.

By exchanging split dCas9 domains according to the claimed invention, sensory switches allow differential regulations on one gene, or activating two different genes in response to cell-type specific microRNAs. Foreseeable variants include combining the sensory switch with other tissue and cellular inputs to enable new approaches for more complex regulations on the Cas9/dCas9 function. Using the claimed invention, split Cas9 system can be delivered in vivo by using recombinant adenovirus-associate viruses (rAAV). The disclosed circuit design principles provided a useful method to reduce the size of synthetic circuits by integrating and swapping split Cas9/dCas9 domains fused with different functional domains. Foreseen variants include combination of the split Cas9/dCas9 system with rAAV delivery systems, Cas9/dCas9 activity can be controlled to edit and regulate endogenous genes in vivo. Such a CRISPR/Cas9 system has particular utility in biomedical applications in which viral delivery vehicles with a restrictive cargo size are preferred.

### BRIEF DESCRIPTION OF THE DRAWINGS

FIG. 1 is a schematic representation of Cas9 with split site locations.

FIG. 2 is a schematic representation of Cas9 split at split site location residues 203-204, 468-469, 713-714 and 1153-1154.

FIG. 3 is a schematic diagram of reconstitution of split Cas9 domains for gene editing.

FIG. 4 is a graphical representation of intein-split and unsplit Cas9 reporter gene expression levels.

FIG. 5 is a schematic diagram of reconstitution of split Cas9 using dCas9:VPR pairs in HEK293.

FIG. 6 is a graphical representation of intein-split and unsplit dCas9 reporter gene expression levels.

FIG. 7 is a schematic illustration of reconstitution of split dCas9 domains for transcriptional control using activation domains.

FIG. 8 is a graphical representation of TagBFP expression.

FIG. 9 is a schematic illustration of the reconstitution of the split dCas9 pair at residue 1153 with EQR mutations in the PI domain.

FIG. 10 is a graphical representation of corresponding fluorescence intensity.

FIG. 11 is a high level schematic illustration of a genetic three input logic AND circuit.

FIG. 12 is a more detailed schematic illustration of a genetic three input logic AND circuit.

FIG. 13 is a graphical representation of TagBFP expression.

FIG. 14 is a more detailed schematic illustration of a genetic three input logic AND circuit.

FIG. 15 is a graphical representation of TagBFP expression illustrating the function of a three-input logic AND circuit using three fragment split-dCas9.

FIG. 16 is a schematic illustration of a two input genetic circuit with one output.

FIG. 17 is a schematic illustration of activation domain optimization.

FIG. 18 is a graphical representation of activation domain optimization.

FIG. 19 is a graphical representation of the ON and OFF states of the sensory switch.

FIG. 20 is a schematic illustration of an alternate two input genetic circuit with one output.

FIG. 21 is a graphical representation of improvements in the ON and OFF states of the sensory switch.

FIG. 22 is a graphical representation of improvements in the ON and OFF states of the sensory switch.

FIG. 23 is a schematic illustration of an alternate two input genetic circuit with one output.

FIG. 24 is a graphical representation of improvements in the ON and OFF states of the sensory switch.

FIG. 25 is a schematic illustration of the optimization of the sensory switch circuit by replacing IntC:dCas9C:VPR with IntC:dCas9C-VRER:VPR.

FIG. 26 is a graphical representation of improvements in the ON and OFF states of the sensory switch.

FIG. 27 is a schematic illustration of a two input with two output genetic sensory switch.

FIG. 28 depicts a schematic representation of the genetic components designed to test orthogonality of the circuit.

FIG. 29 is a graphical representation of the orthogonality test.

FIG. 30 is a graphical representation of genetic circuit ‘leakiness’.

FIG. 31 is an additional graphical representation of genetic circuit ‘leakiness’.

FIG. 32 is a schematic representation of sensory switches for exchanging different dCas9 activation domains.

FIG. 33 is an alternate schematic representation of sensory switches for exchanging different dCas9 activation domains.

FIG. 34 is a preferred embodiment of a schematic representation of sensory switches for exchanging different dCas9 activation domains.

FIG. 35 is a graphical representation of greatly reduced genetic circuit ‘leakiness’.

FIG. 36 is a schematic representation of genetic circuitry for identification of bladder cancer utilizing split dCas9 domains.

FIG. 37 is a graphical representation of hTERT and hupII individual and in combination reflecting Blue Fluorescent Protein activity of the bladder cancer detection genetic circuitry.

FIG. 38 is a schematic representation of genetic circuitry with a plurality of inputs and receptors utilizing split dCas9 domains.

FIG. 39 is a schematic representation of genetic circuitry for tissue specific activation gene E1A utilizing split dCas9 domains delivered by adenovirus and then controlling replication of adenovirus.

FIG. 40 is a schematic representation of genetic circuitry for plant cell modification utilizing split dCas9 domains.

FIG. 41 is a schematic representation of genetic circuitry for plat cell gene editing utilizing split Cas9 domains.

### DETAILED DESCRIPTION OF THE INVENTION

FIG. 1 is a schematic representation of Cas9 (100) with pair 1-4 split site locations (111, 112, 113, 114) to aid in illustrating functional reconstitution of split Cas9 domains. According to the Cas9 sequence and structural information the split sites are selected where serine is at the +1 amino acid position when fused to the C-terminal Intein fragment. All four selected split sites are surface residues and located in the loop region, which can be more accessible for intein trans-splicing reaction and have less effect on the protein folding. In the illustrative example, eight pairs of split Cas9 constituents are constructed that either fuse to the N-terminal (IntN) and C-terminal (IntC) split inteins or not. The Cas9-DNA targeting specificity is determined by both the Cas9-associated guide RNA (gRNA) and a short protospacer adjacent motif (PAM) directly downstream of the DNA recognition site. The Streptococcus pyogenes Cas9 (SpCas9) protein usually consists of a recognition lobe and a nuclease lobe. The recognition lobe contains a bridge helix at residues 60-93 (103), a REC1 domain at residues 94-179 and 308-713 (105, 109) and a REC2 domain at residues 180-307 (107), while the nuclease lobe includes a RuvC domain at residues 1-59, 718-769, and 909-1098 (101,117,121), a HNH domain at residues 775-908 (119) and a PAM-interacting (PI) domain at residues 1099-1368 (125). In one embodiment of the invention, Cas9 is intein split at residues 203-204, 468-469, 713-714 and 1153-1154.

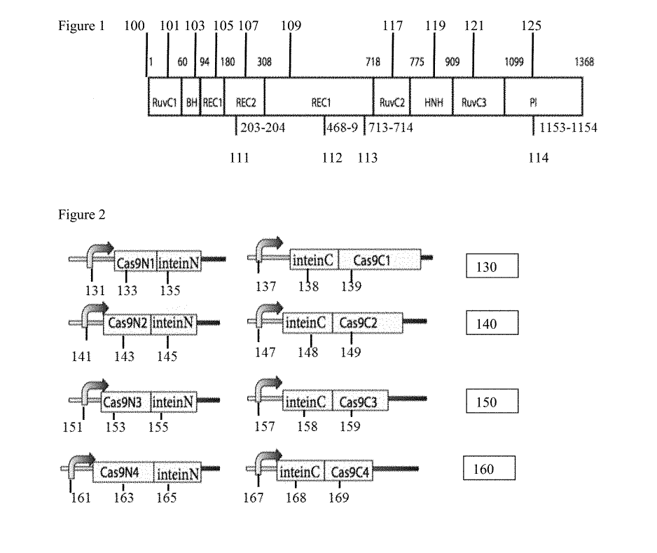

FIG. 2 is a schematic representation of Cas9 split at split site location residues 203-204, 468-469, 713-714 and 1153-1154. Depending upon the split site, four different Cas9 pairs (130, 140, 150, 160) are created. Split pair I (130) is created by splitting at residue site 203-204 resulting in first split half promoter (131) first split half Cas9N (133) and inteinN portion (135) as well as second split half promoter (137) inteinC (138) and Cas9C (139). Split pair II (140) is created by splitting at residue site 468-469 resulting in first split half promoter (141) first split half Cas9N (143) and inteinN portion (145) as well as second split half promoter (147) inteinC (148) and Cas9C (149). Split pair III (150) is created by splitting at residue site 713-714 resulting in first split half promoter (151) first split half Cas9N (153) and inteinN portion (155) as well as second split half promoter (157) inteinC (158) and Cas9C (159). Split pair IV (160) is created by splitting at residue site 1153-1154 resulting in first split half promoter (161) first split half Cas9N (163) and inteinN portion (165) as well as second split half promoter (167) inteinC (168) and Cas9C (169). In a complementary embodiment, split Cas9 fragments across different split pairs yield combinations that provided the complete polypeptide sequence activate gene expression even when fragments are partially redundant.

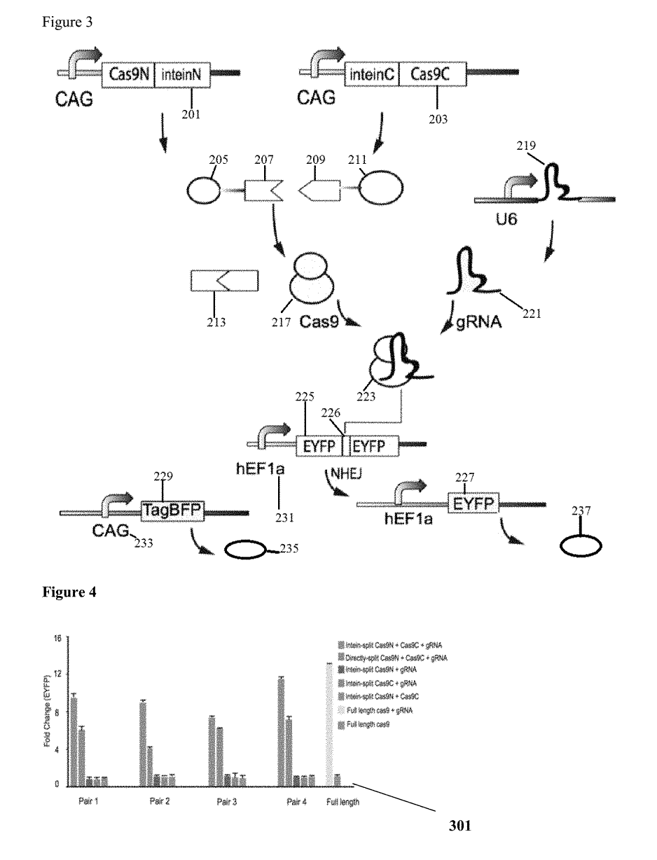

FIG. 3 is a schematic diagram of reconstitution of split Cas9 domains for gene editing. During reconstruction, intein split Cas9 first portion (201) and second portion (203) are recombined where Cas9N (205) and Cas9C (211) are joined through the conjugation of first intein portion (207) and second intein portion (209) resulting in combined separate intein (213) and complete Cas9 (217). In an illustrative embodiment, a repeat sequence is inserted (226) in the middle of enhanced yellow fluorescent protein (EYFP) reporter gene (225). Through expression (219) of guide RNA (221) creating a fully functional gRNA+Cas9 complex (223), the functional Cas9 protein cleaves the EYFP repeat region, triggering the reconstitution of inactive EYFP into the full-length active EYFP reporter gene (225) resulting in EYFP expression (237) as a result of promoter hEF1α (231).

FIG. 4 is a graphical representation (301) of intein-split and unsplit Cas9 reporter gene expression levels. By using this EYFP-reconstitution reporter system, all four intein-mediated split-Cas9 pairs efficiently reactivated the EYFP expression in a human embryonic kidney HEK293 cells. The Cas9 sets split at residues 203, 468, 713 and 1153 without intein fusion, displayed a reduced activity compared to their counterparts with appropriate intein fusions.

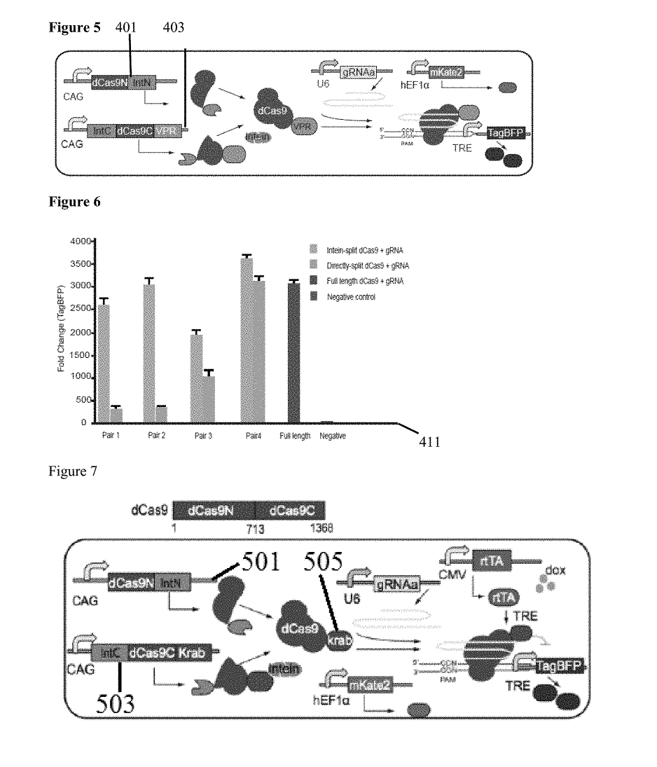

FIG. 5 is a schematic diagram of reconstitution of split Cas9 using dCas9:VPR pairs (401, 403) in HEK293. In this illustrative embodiment, a similar set of split dCas9:VPR pairs recapitulate the function of the full-length dCas9:VPR in HEK293 cells by transient transfection.

FIG. 6 is a graphical representation (411) of intein-split and unsplit dCas9 reporter gene expression levels. Three of split pairs without intein fusions showed a reduced activation function compared to their counterparts with intein fusions. In contrast, the dCas9 protein directly split at position 1135 was almost as active as the intact dCas9 protein. Three of the split dCas9:VPR pairs fused to intein fragments activated the reporter gene as efficiently as the full-length dCas9:VPR, while the dCas9:VPR split at residue 713 was not as efficient, indicating that the VPR fusion and the choice of split site might affect reconstitution of split dCas9 fragments and then influence the protein activity.

FIG. 7 is a schematic illustration of reconstitution of split dCas9 domains (501, 503) for transcriptional control using activation domains (505). Cas9 splitting is further complemented through the addition of a variety of regulatory domains. In the illustrative example, functional reassembly is obtained from dCas9 constituents (501, 503) split at either residue 713 or residue 1153 when fused to different transcription regulatory domains, such as Krab (505), Suntag and VP64 (not shown).

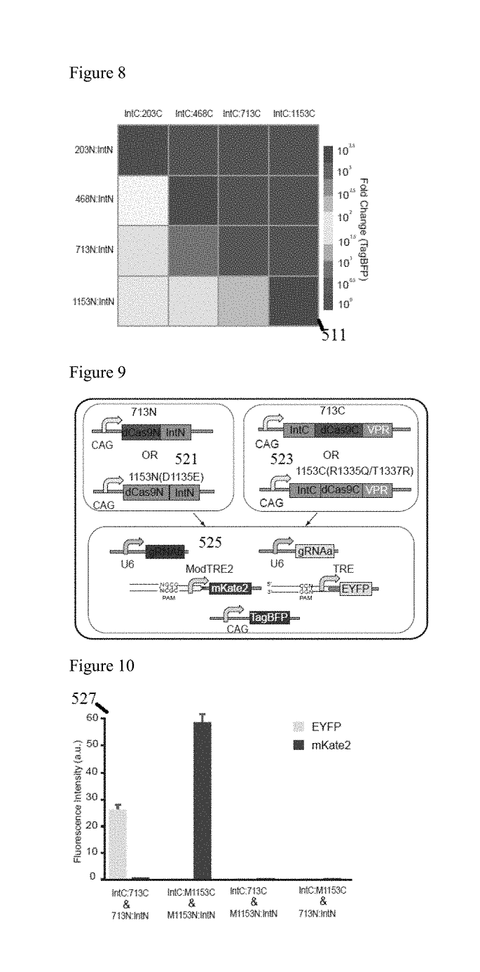

FIG. 8 is a graphical representation (511) of TagBFP expression indicating activity of split dCas9 fragments across different split pairs when reconstituted into a complete dCas9. While combinations of dCas9 IntN and IntC fragments that resulted in incomplete dCas9 proteins failed to activate TagBFP expression, fragment combinations that provided the complete polypeptide sequence activated TagBFP expression even when the two fragments were partly redundant. It is noteworthy that the dCas9 pair split at residue 1153 divided the PI domain into two fragments.

FIG. 9 is a schematic illustration of the reconstitution (525) of the split dCas9 pair (521, 523) at residue 1153 with EQR mutations in the PI domain. In this illustrative example, orthogonality of the split set at residue 1153 by introducing EQR mutations in the PI domain is explored.

FIG. 10 is a graphical representation (527) of corresponding fluorescence intensity. This figure illustrates that the reconstitution of the split dCas9 pair at residue 1153 with the EQR mutations only activates the mKate2 reporter gene with the NGCG PAM but not the EYFP reporter gene with the NGG PAM, while the reconstitution of the split dCas9 pair at residue 713 without mutations leads to the opposite results. In addition, no cross activity is found when either the wild-type N-terminal or C-terminal dCas9 fragment is combined with the EQR mutant C-terminal or N-terminal dCas9 constituents. As a direct and intended consequence, these orthogonal split dCas9 pairs have particular utility and applicability in the construction of complex genetic circuits and logic gates.

Construction of Three-Input Logic AND Circuit

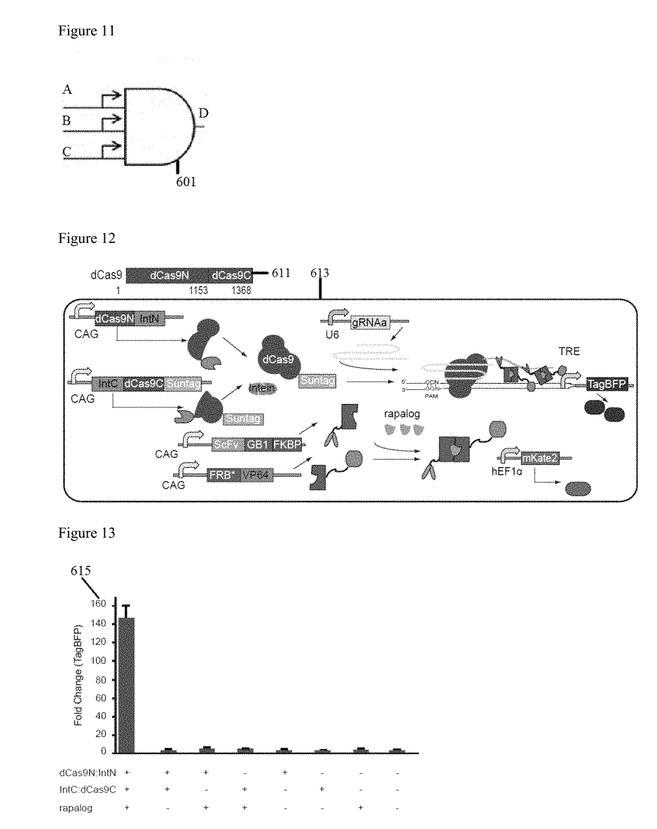

FIG. 11 is a high level schematic illustration of a genetic three input logic AND circuit (601). In the following embodiment of the invention, a three input logic AND genetic circuit (601) is created by building upon the orthogonal split dCas9 sections. In the corresponding truth table for a three input logic AND circuit, all three inputs need to be expressed in order to generate an output.

TABLE 1
Three-input logic AND circuit truth table

| Input A | Input B | Input C | Output D |
|---|---|---|---|
| 0 | 0 | 0 | 0 |
| 0 | 0 | 1 | 0 |
| 0 | 1 | 0 | 0 |
| 0 | 1 | 1 | 0 |
| 1 | 0 | 0 | 0 |
| 1 | 0 | 1 | 0 |
| 1 | 1 | 0 | 0 |
| 1 | 1 | 1 | 1 |

FIG. 12 is a more detailed schematic illustration of a genetic three input logic AND circuit (613). Split key (611) illustrates the dCas9 split details to enable logic AND circuit (613) by using the dCas9 constituents split at residue 1153 and the Suntag repetitive peptide scaffold that contains ten ScFv binding motifs. In the illustrative embodiment, the ScFv along with a small solubility tag GB1 and VP64 fragments are respectively fused to FK506 binding protein 12 (FKBP) and FKBP rapamycin binding (FRB*) domains with a T2089L mutation derived from the mammalian target of rapamycin (mTOR). As a direct and intended consequence, the resulted fusion proteins, ScFv:GB1:FKBP and FRB*:VP64 form a heterodimer in the presence of the rapamycin analog AP21967 (rapalog).

FIG. 13 is a graphical representation (615) of TagBFP expression illustrating the function of a three-input logic AND circuit using split-dCas9 constituents and rapalog in HEK293 cells. FIG. 6(c) illustrates that the logic AND circuit operates correctly in response to all eight different combinations of three inputs with an ON/OFF ratio greater than 140-fold.

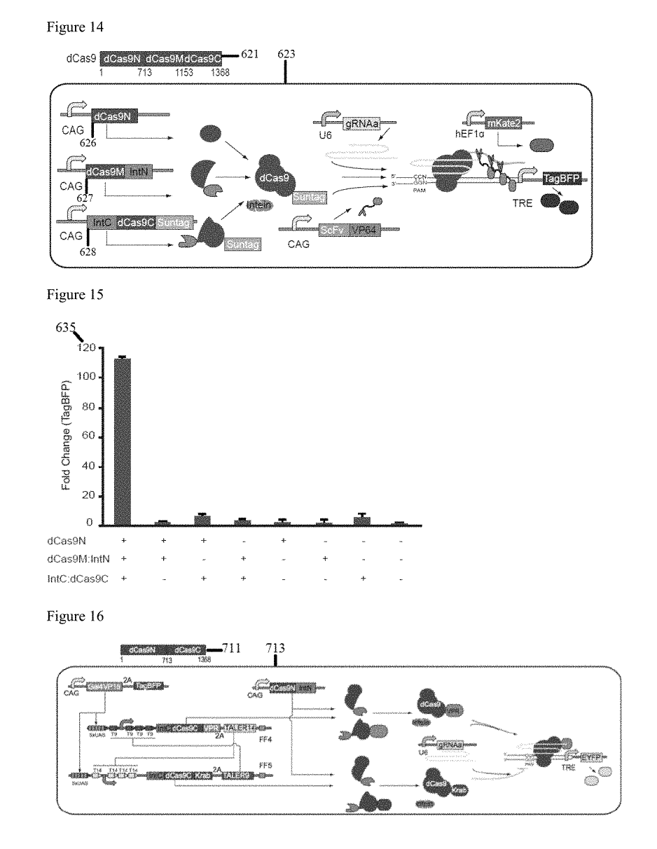

FIG. 14 is a more detailed schematic illustration of a genetic three input logic AND circuit (613). Split key (621) illustrates the dCas9 split details to enable logic AND circuit (623). As previously illustrated, dCas9:VPR pairs split at residues 713 and 1153 without intein more efficiently activated the expression of TagBFP than the other two split pairs. As a result, in this illustrative embodiment a three-input logic AND circuit is created by splitting dCas9 into three fragments (626, 627, 628), including dCas9N (626) containing dCas9 residues from 1 to 713, dCas9M:IntN (627) containing the residues from 714 to 1153, and IntC:dCas9C:Suntag (628) containing the residues from 1154 to 1368.

FIG. 15 is a graphical representation (635) of TagBFP expression illustrating the function of a three-input logic AND circuit using three fragment split-dCas9. In this illustrative embodiment, the split-dCas9 logic AND circuit induces TagBFP expression greater than 110-fold only when all three split-dCas9 constituents were added. While the illustrative embodiment is show as applied in HEK293 cells, the choice of cell lines is illustrative only and not by way of limitation and can be broadly applied in a variety of cell line applications.

Two-Input and One-Output Sensory Switch

FIG. 16 is a schematic illustration of a two input genetic circuit with one output. FIG. 7a illustrates a TALER sensory switch controlled by two different shRNAs/microRNAs. Split key (711) illustrates the dCas9 split details to enable the two-input logic circuit (713) with one output. In the disclosed embodiment, to illustrate the domain exchange of dCas9 constituents IntC:dCas9C:VPR and IntC:dCas9C:Krab is fused to TALER14 and TALER9 respectively, which reconstituted with a constitutive dCas9N:IntN to activate or repress the expression of the EYFP reporter gene by competitively binding to the TRE promoter.

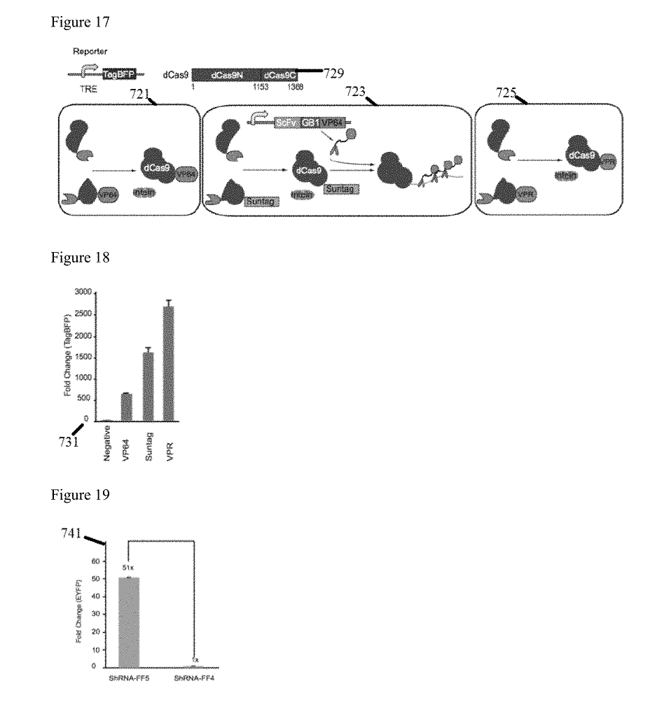

FIG. 17 is a schematic illustration of activation domain optimization in which different activation domains VP64 (721), Suntag (723) and VPR (725) are fused to dCas9C. As depicted in split key (729), the dCas9 constituents are split at residue 1153. TagBFP was used as the reporter gene.

FIG. 18 is a graphical representation (731) of activation domain optimization. Each bar shows mean fold changes (mean±SEM; n=3) of TagBFP fluorescence measured by using flow cytometry 48 hours after transfection in HEK293 cells. The VPR activation domain is chosen in the illustrative embodiment because the activation efficiency is greater than both VP64 and Suntag activation domains.

FIG. 19 is a graphical representation (741) of the ON and OFF states of the sensory switch, illustrating control of the states of the sensory switch by shRNA-FF4 or shRNA-FF5. The shRNA-FF5 and shRNA-FF4 respectively triggered the ON and OFF states of the sensory switch a ON/OFF ratio of 51-fold.

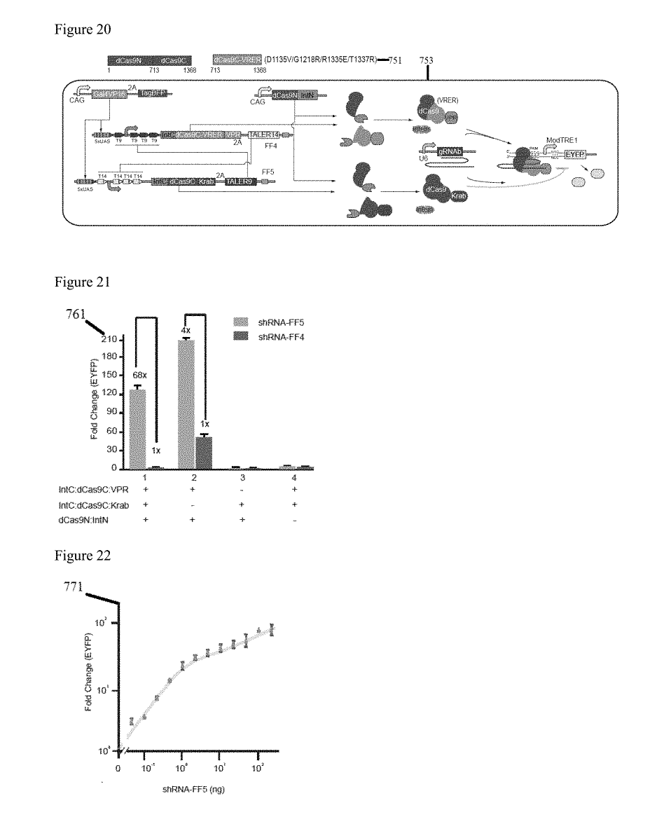

FIG. 20 is a schematic illustration of an alternate two input genetic circuit with one output. Split key (751) illustrates the dCas9 split details to enable the two-input logic circuit (753) with one output. To further improve the performance of sensory switch in the disclosed embodiment, IntC:dCas9C-VRER:VPR is used that contains a mutant PI domain (D1135V/G1218R/R1335E/T1337R) to switch the PAM recognition specificity of the reconstituted dCas9 from NGG to NGCG. Accordingly, a modified TRE promoter (ModTRE1) is constructed that contains 7 gRNAb binding sites with the NGCG PAM sequences upstream of the minimal CMV promoter, followed by three gRNAb binding sites with the NGG PAM sequences.

FIG. 21 is a graphical representation (761) of improvements in the ON and OFF states of the sensory switch. The results illustrate that the ON/OFF ratio of this modified sensory switch increased to 68-fold. In contrast, the shRNA-FF4 failed to efficiently repress the EYFP expression in the absence of the feedback regulation exerted by the 2A-linked IntC:dCas9C:Krab and TALER9.

FIG. 22 is a graphical representation (771) of improvements in the ON and OFF states of the sensory switch. As illustrated, the sensory switch responds to the shRNA-FF5 input in a dosage dependent manner.

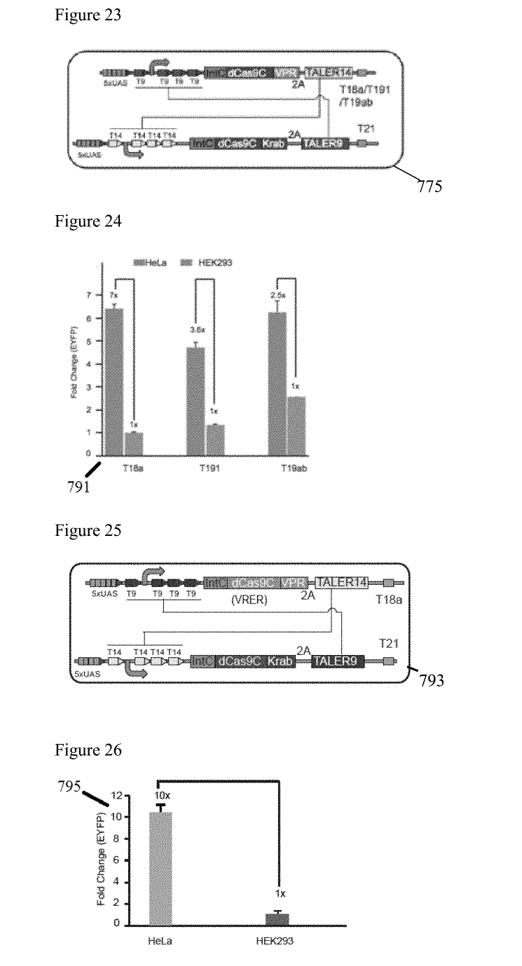

FIG. 23 is a schematic illustration (775) of an alternate two input genetic circuit with one output illustrating setting states of sensory switches by endogenous microRNAs. For simplicity, only the core of the sensory switch is shown. In this illustrative embodiment, cell-type specific microRNAs are connected to control the sensory switch by fusing four tandem repeats of fully complementary microRNA binding sites in the 3′-UTR of the IntC:dCas9C:VPR-2A-TALER14 and IntC:dCas9C:Krab-2A-TALER9. In this embodiment, miR18a, miR191, miR19a-3p and miR19b-3p that are highly expressed in HEK293 cells but not in HeLa can be used as the HEK293 specific microRNA markers, while miR21 can be used as the HeLa specific microRNA marker.

FIG. 24 is a graphical representation (791) of improvements in the ON and OFF states of the sensory switch. Data shown as the mean fold change ±SEM; n=3) of EYFP fluorescence, measured 48 h after transfection. After transfection into HeLa and HEK293 cells, the sensory switch responds correctly to miR21/miR18a, miR21/miR191 and miR21/miR19ab (a composite marker that includes both miR19a-3p and miR19b-3p) with a ON/OFF ratio of 7-fold, 3.6-fold and 2.5-fold respectively. As a direct and intended consequence, the sensory switch can recapitulate the function of either dCas9:VPR or dCas9:Krab in response to two different endogenous microRNAs.

FIG. 25 is a schematic illustration (793) of an alternate two input genetic circuit with one output illustrating the optimization of the sensory switch circuit by replacing IntC:dCas9C:VPR with IntC:dCas9C-VRER:VPR, with FIG. 26 illustrating (795) the increase of the ON/OFF ratio to 10-fold in response to miR21/miR18a input combination. As a direct and intended consequence of the illustrative embodiment, the sensory switch responds to the miR21 input in a dosage dependent manner.

Two-Input and Two-Output Sensory Switch

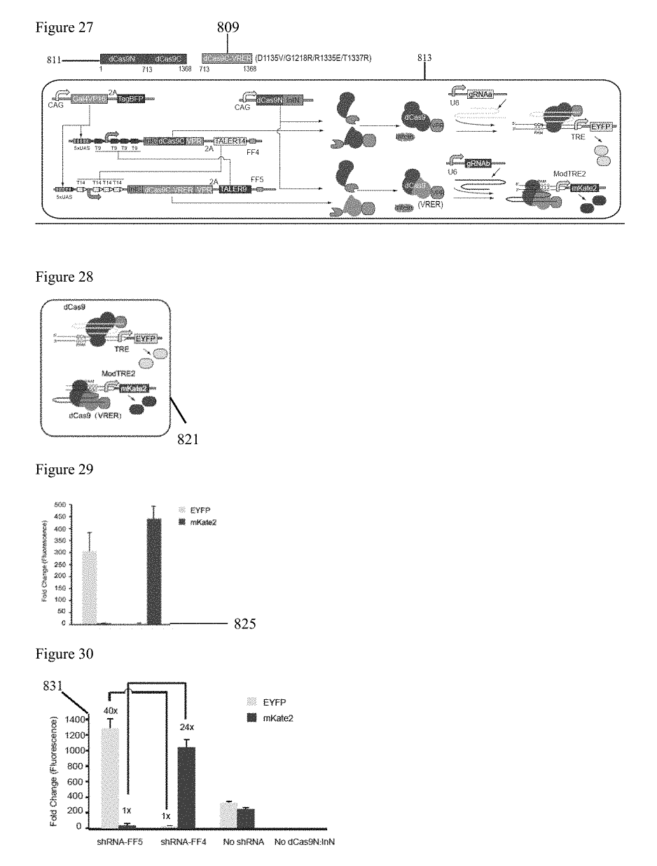

FIG. 27 is a schematic illustration of a two input with two output genetic sensory switch. Split key (801) illustrates the dCas9 split details utilized in the two-input genetic logic circuit (803) with two outputs. In this illustrative example, logic circuit (803) is a schematic representation of a two-input and two-output sensory switch implemented by swapping split dCas9 domains that recognize two different PAM sequences. The dCas9 constituents are split at residue 713. The second rectangle (809) in split key (801) represents the mutant dCas9 domain (VRER) that can recognize the NGCG PAM sequences but not the NGG PAM sequences. The ModTRE2 promoter contains 7 repeats of gRNAb binding sites with the NGCG PAM sequences upstream of a miniCMV core. This illustrative embodiment tests whether the sensory switch can be used to activate two different output genes in response to two different shRNAs by replacing the IntC:dCas9C:Krab with the orthogonal activator IntC:dCas9C-VRER:VPR.

In this illustrative embodiment, FIG. 28 depicts a schematic representation (821) of the genetic components designed to test orthogonality of the circuit. In this illustrative embodiment, the dCas9 can recognize the NGG PAM sequence, and then activate EYFP. The mutant dCas9 that contains 4 point mutations (D1135V/G1218R/R1335E/T1337R, or VRER in short) can recognize the NGCG PAM sequence, and then activate mKate2.

FIG. 29 is a graphical representation (825) of the orthogonality test. The orthogonality test shows that in this illustrative embodiment, IntC:dCas9C-VRER:VPR only activated the modified TRE promoter (ModTRE2) with the NGCG PAM sequences but not the original TRE promoter with the NGG PAM sequences. Graphical representation (825) shows the fluorescence intensity of EYFP and mKate2 in a co-transfection example. Each bar shows mean fold change (mean±SEM; n=3) of EYFP or mKate2 fluorescence measured by using flow cytometer 48 h after transfection in HEK293 cells.

Circuit ‘Leakiness’ Example

FIG. 30 is a graphical representation (831) of genetic circuit ‘leakiness’ setting states of the illustrative sensory switch by artificial shRNA-FF5 and shRNA-FF4. The shRNA-FF5 and shRNA-FF4 respectively induce a high level of EYFP and mKate2 with a greater than 20-fold ON/OFF ratio, although this illustrative example indicates a leaky expression of both EYFP and mKate2 at the OFF state.

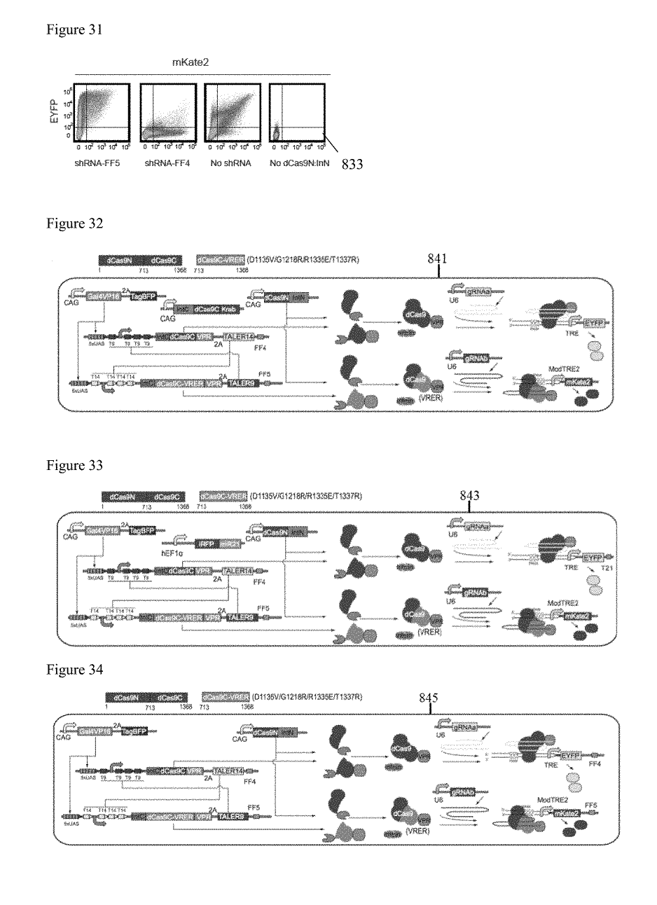

FIG. 31 is an alternate graphical representation (833) of genetic circuit ‘leakiness’ in scatter plot format wherein each scale bar in images represents 50 μm. In the illustrative example, the EYFP level gradually decreases when increasing the amount of shRNA-FF4, while the mKate2 level increases in a shRNA-FF4 dosage dependent manner.

Solution to Circuit ‘Leakiness’

Because dCas9-VRER:VPR only activated the modified TRE promoter (ModTRE2) with the NGCG PAM sequences but not the original TRE promoter with the NGG PAM sequences as illustrated in FIGS. 8(b) and 8(c), the illustrative example indicated that the leaky expression may be due to the trace of split dCas9 activation domains at the OFF state of the sensory switch.

FIG. 32 is a schematic representation (841) of sensory switches for exchanging different dCas9 activation domains. In this embodiment to reduce the leaky expression of EYFP in the OFF state, a trace of dCas9:Krab is reconstituted to exert a weak transcriptional repression on the EYFP expression.

FIG. 33 is an alternate schematic representation (843) of sensory switches for exchanging different dCas9 activation domains. In this alternate embodiment, four tandem repeats of miR21 target sites were fused to the 3′-UTR of the EYFP reporter gene. The schematic illustrates the adding of a trace of miR21 to apply a weak post-transcriptional repression on the EYFP expression. Applying both a weak transcriptional repression by the dCas9-Krab and a weak post-transcriptional repression by exogenously introducing miR21 can greatly reduce the leaky expression of EYFP, although the EYFP level at the ON state also decreases.

FIG. 34 is a preferred embodiment of a schematic representation (845) of sensory switches for exchanging different dCas9 activation domains. In FIG. 8(h), the EYFP reporter gene is fused with four tandem repeats of FF4 target sites in the 3′-UTR and the mKate2 gene is fused with four tandem repeats of FF5 target sites in the 3′-UTR. The feed-forward loop is a useful circuit architecture to reduce expression leakiness. By fusing four tandem repeats of shRNA-FF4 target site in the 3′-UTR of EYFP and four tandem repeats of shRNA-FF5 target site in the 3′-UTR of the mKate2, shRNA-FF4 and shRNA-FF5 respectively repress EYFP and mKate2 through a feed-forward loop, effectively solving genetic circuit ‘leakiness’ as an intended and direct consequence of the illustrative embodiment.

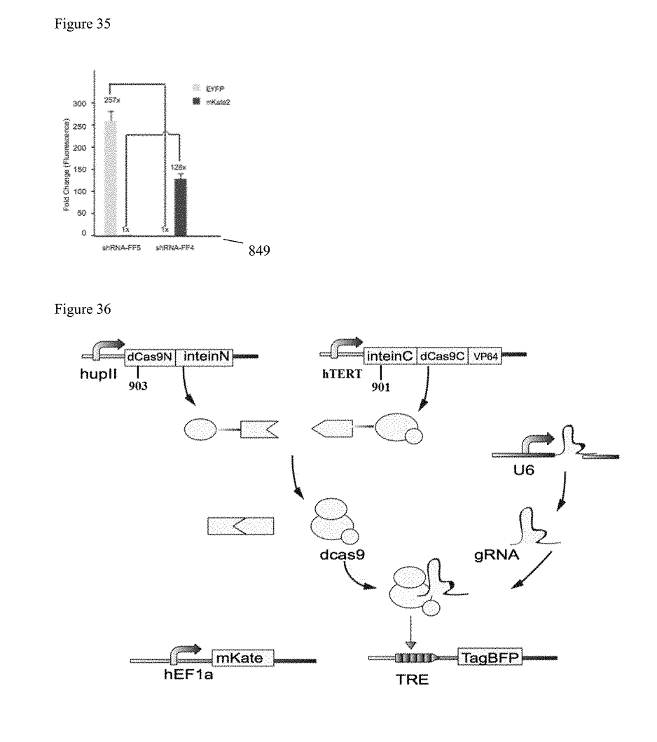

FIG. 35 is a graphical representation (849) of greatly reduced genetic circuit ‘leakiness. As a direct and intended use of the feed-forward loop according to the claimed invention, very little leaky expression of either EYFP or mKate2 is observed.

FIG. 36 is a schematic representation of hTERT and hupII genetic circuitry for identification of bladder cancer utilizing split dCas9 domains (901, 903). In this embodiment of the presently claimed invention, identification of bladder cancer cells is enabled by use of split dCas9 to incorporate the bladder cancer and cancer-specific promoter hupII as well as hTERT. With intein split dCas9 in the present embodiment, transfection difficulties are reduced while increasing the efficiency of gene editing, expression and regulation. Furthermore, tumor cell-specific binding of the promoter, and as well as use of logic gates are implemented to achieve the detection of tumor cells, to improve the specificity of cell identification. The bladder cancer detection embodiment described is by way of illustration rather than limitation as it can additionally take advantage of inducible promoters such as TRE to express gRNA, and in turn, can be topical dox-induced with greater control and specificity.

In the illustrative embodiment, split inactivated Cas9 protein is used in bladder cancer cell-specific detection verification. dCas9 used are derived from spdCas9. The recombinant vector is constructed as follows: pENTR_L4_hupII_L1, wherein the first 745-1100 encodes hupII promoter; pENTR_L4_hTERT_L1, wherein bp705-1160 encodes hTERT promoter; dCas9N-InteinN-phupII, wherein base pairs 4843-7279 encode a fusion protein dCas9N-InteinN encoding gene, wherein base pairs 4284-4637 encode hupII promoter, the vector expressing a fusion protein dCas9N-InteinN. The fusion protein dCas9N-InteinN is described sequentially from the upstream in the dCas9N InteinN composition. phTERT-InteinC-dCas9C-VP64, wherein base pairs 3421-5715 encode a fusion protein InteinC-dCas9C-VP64 encoding gene, base pairs 2798-3253 encode promoter hTERT, the vector expressing a fusion protein InteinC-dCas9C-VP64.

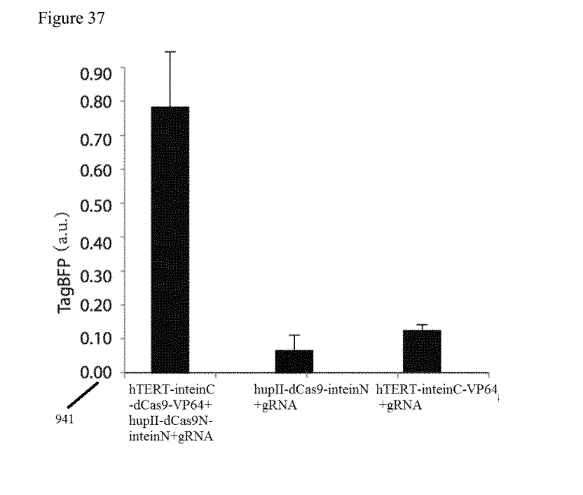

Demonstration of the use of split inactivated Cas9 protein for bladder cancer cell-specific detection verification. Results are indicated through the use of reporter proteins. TagBFP is a reporter protein which can be detected by standard luminescence intensity detection equipment. In normal cells which lack the specific promoter, tre does not start and TagBFP will not light. In bladder cancer cell lines with the specific promoter, tre starts and TagBFP expression will begin resulting in detectable luminescence. Use of TagBFP is by illustration only and may be replaced with other fluorescent proteins. Bladder cancer cell-specific detection takes place by way of genetic circuitry utilizing hupII and hTERT. Using these two promoters and split cas9 system bladder cancer detection takes place as follows: After the split, reorganization dCas9 takes place together with gRNA and will focus on TRE promoter upstream of the respective sites. dCas9 fusion VP64 can activate the TRE promoter, expressing TagBFP fluorescent protein. If split recombination does not occur, and only the dCas9N end or dCas9C end of the expression is present, VP64 will not be present at the TRE appropriate sites and expression of the downstream reporter gene will not activate. The illustrative bladder cancer cell detection embodiment is constructed using the phupII-dCas9N-InteinN plasmid, phTERT-InteinC-dCas9C-VP64 plasmid, pU6-Guide RNA1 plasmid, pEF1a-mKate plasmid (internal control plasmid), pTRE-TagBFP plasmids. Illustrative data is provided in FIG. 37 representing transfection into bladder cancer 5637 cell line from Shanghai Su Seoul biological Technology Co., Ltd. at 100 ng per well for each plasmid transfection. In order to verify the accuracy of the illustrative embodiment, dCas9N-InteinN alone as well as only InteinC-dCas9C-VP64 are provided as negative controls (Table 2 in 3 groups).

TABLE 2
hupII/hTERT plasmid transfection

| | Co-transfection using plasmid\amount (ng) | | |
|---|---|---|---|
| | 1 | 2 | 3 |
| phupII-dCas9N-InteinN | 100 | 100 | |
| phTERT-InteinC-dCas9C- | 100 | | 100 |
| VP64 | | | |
| pU6-guide RNA1 | 100 | 100 | |
| pTRE-TagBFP | 100 | 100 | 100 |
| pEF1a-mKate | 100 | 100 | 100 |
| pDT7004 | | 100 | 100 |

FIG. 37 is a graphical representation (941) of hTERT and hupII individual and in combination reflecting Blue Fluorescent Protein activity of the bladder cancer detection genetic circuitry transfection results. Transfection occurs 48 hours after flow cytometry analysis, testing mKate fluorescence intensity and TagBFP fluorescence intensity. In this embodiment of the invention, transfection results are shown. TagBFP fluorescence is used to measure the relative strength of the split Cas9 system efficiency on bladder cancer cell-specific detection. In addition, mKate is used as a reference for the calibration of co-transfection efficiency. As derived from the illustration (941), TagBFP fluorescence relative intensity=experimental group TagBFP fluorescence intensity/the same group mKate fluorescence intensity. The individual columns reflect hTERT-InteinC-dCas9C-VP64+hupII-dCas9N-InteinN+gRNA from Table 2 in the first column; hupII-dCas9N-InteinN+gRNA from Table 2 in the second column, and hTErT-InteinC-dCas9C-VP64+gRNA Table 2 in the third column. In this embodiment of the invention, TagBFP relative fluorescence intensity of 0.78 (hTERT-InteinC-dCas9C-VP64+hupII-dCas9N-InteinN+gRNA), can be seen in the split dCas9 regulatory proteins which far greater than the control group TagBFP relative fluorescence intensity (less than 0.10).

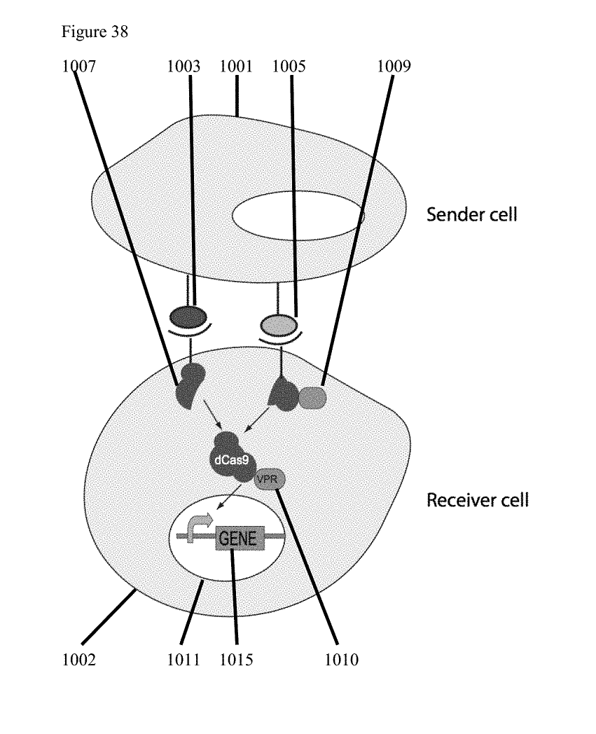

FIG. 38 is a schematic representation of genetic circuitry with a plurality of inputs and receptors utilizing split dCas9 domains. In this illustrative example, targeted gene 1015 control and regulation takes place by sender cell 1001 presentation of first messenger signal 1003 and second messenger signal 1005. Genetic regulation takes place in receiver cell 1002 when first cellular receptor 1007 and second cellular receptor 1009 are activated to form dCas9 complex with VPR 1010. Genetic regulation takes place when dCas9 complex 1010 passes through nucleus 1011 to regulate genetic target 1015.

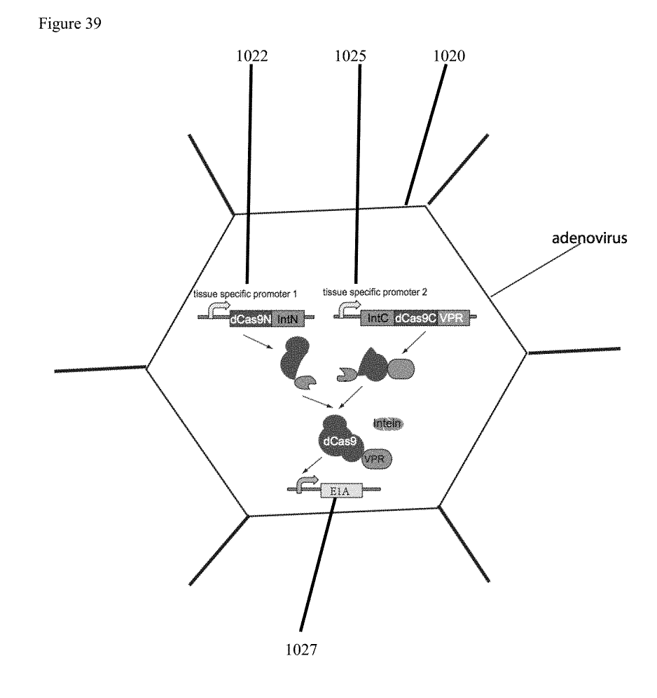

FIG. 39 is a schematic illustration of genetic circuitry for tissue specific activation of adenovirus' replication utilizing split dCas9 domains. In this tissue specific illustrative example, adenovirus 1020 is used to deliver first tissue specific split dCas9 complex 1022 and second tissue specific split dCas9 complex 1025 which assemble in the target tissue (not shown) to selectively control gene target E1A 1027. E1A decides the replication of adenovirus.

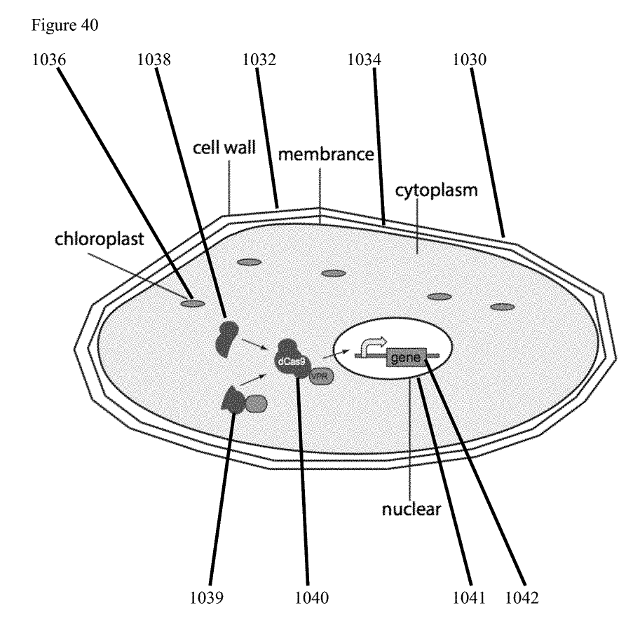

FIG. 40 is a schematic representation of genetic circuitry for plant cell modification utilizing split dCas9 domains. In this plant specific illustrative example, plant cell 1030 containing chloroplast 1036 is modified through the plant cell wall 1032 and cell membrane 1034. First dCas9 domain 1038 and second dCas9 domain 1039 assemble to form fully functioning dCas9 complex 1040. Fully activated Cas9 complex 1040 passes through plant cell nucleus 1041 to perform plant nuclear DNA control 1042. Reference to ‘first’ and ‘second’ are by way of illustration and not by limitation as the illustrative example may be expanded to accommodate a plurality of split dCas9 portions to accomplish plant cell modification.

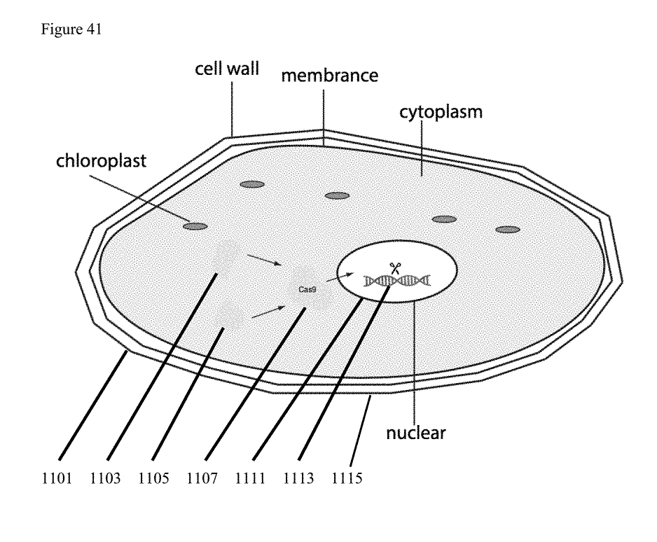

FIG. 41 is a schematic representation of genetic circuitry for plant cell modification utilizing split Cas9 domains. In this plant specific illustrative example, plant cell 1101 is modified through the cell wall 1115. First Cas9 domain 1103 and second Cas9 domain 1105 assemble to form fully activated Cas9 complex 1107. Fully activated Cas9 complex 1107 passes through plant cell nucleus 1111 to perform plant nuclear DNA cutting 1113. Reference to ‘first’ and ‘second’ are by way of illustration and not by limitation as the illustrative example may be expanded to accommodate a plurality of split Cas9 portions to accomplish plant cell modification.

We claim:

1. A non-naturally occurring split Cas9 protein system comprising:
a combination of a plurality of Cas9 portions which are intein split, wherein the split sites were surface residues and located in the loop region of Cas9 and
each Cas9 portion is capable of fusion to a transcription regulatory domain.

2. The non-naturally occurring split Cas9 protein system of claim 1 wherein said split sites are selected from the split site locations consisting of Cas9 splits at residues 203, 468, 713 and 1153.

3. The non-naturally occurring split Cas9 protein system of claim 2 additionally comprising transcription regulatory domains selected from the group consisting of Krab, VPR, Suntag and VP64.

4. The non-naturally occurring split Cas9 protein system of claim 2 additionally comprising split Cas9 portions across different split pairs to yield combinations that provided the complete polypeptide sequence activate gene expression even when fragments are partially redundant.

5. The non-naturally occurring split Cas9 protein system of claim 2 additionally comprising genetic circuitry components with hupII and HTERT promoters operably connected to an indication component.

6. The non-naturally occurring split Cas9 protein system of claim 5 wherein said indication component is TagBFP which is operably expressed in the presence of bladder cancer cells.

7. The non-naturally occurring split Cas9 protein system of claim 4 additionally comprising a feed forward loop operably connected to said Cas9 portions.

8. The non-naturally occurring split Cas9 protein system of claim 1 wherein said Cas9 is dCas9.

9. The non-naturally occurring split Cas9 protein system of claim 2 wherein said Cas9 additionally comprises a plurality of genetic logic input components to form an AND circuit utilizing VPR to control gene regulation upon cellular presentation and identification of a plurality of cellular signal components.

10. The non-naturally occurring split Cas9 adenovirus modification system of claim 2 wherein Cas9 additionally comprises a plurality of genetic logic input components to form an AND circuit utilizing VPR to control E1A expression upon tissue specific promoters.

11. A method of providing a cell with a Cas9 protein comprising:
A providing step providing to the cell a first nucleic acid encoding a first portion of the Cas9 protein and a second nucleic acid encoding a second portion of the cas9 protein wherein the first nucleic acid encodes a first portion of the Cas9 protein having a first split-intein and wherein the second nucleic acid encodes a second portion of the Cas9 protein having a second split-intein, wherein the split sites were surface residues and located in the loop region,
a first expression step, wherein the cell expresses the first nucleic acid encoding the first portion of the Cas9 protein,
a second expression step wherein the cell expresses the second nucleic acid encoding the second portion of the Cas9 protein, and
a combination step wherein the first portion of the Cas9 protein and the second portion of the Cas9 protein are joined together to form the Cas9 protein.

12. The method of claim 11 wherein the first portion of the Cas9 protein is the N-terminal lobe of the Cas9 protein up to amino acid 203 and the second portion of the Cas9 protein is the C-terminal lobe of the Cas9 protein beginning at 204.

13. The method of claim 11 wherein the first portion of the Cas9 protein is the N-terminal lobe of the Cas9 protein up to amino acid 468 and the second portion of the Cas9 protein is the C-terminal lobe of the Cas9 protein beginning at 469.

14. The method of claim 11 wherein the first portion of the Cas9 protein is the N-terminal lobe of the Cas9 protein up to amino acid 713 and the second portion of the Cas9 protein is the C-terminal lobe of the Cas9 protein beginning at 714.

15. The method of claim 11 wherein the first portion of the Cas9 protein is the N-terminal lobe of the Cas9 protein up to amino acid 1153 and the second portion of the Cas9 protein is the C-terminal lobe of the Cas9 protein beginning at 1154.

16. The method of claim 11 wherein said cas9 protein is a protein selected from the group consisting of an enzymatically active Cas9 protein, a Cas9 protein nickase and a nuclease null Cas9 protein.

17. The method of claim 11 wherein said providing step additionally comprises providing said first portion and second portion of the Cas9 protein to a human cell line to express a diagnostic indicator resulting from interaction by said Cas9 protein with microRNA from said human cell line.

18. The method of claim 11 wherein said providing step additionally comprises providing said first portion and second portion of the Cas9 protein via recombinant adeno-associated virus(rAAV) to a subject in need via intravenous administration.

19. A non-naturally occurring split Cas9 plant cell modification system comprising:
a combination of a plurality of Cas9 portions which are intein split, wherein the split sites were surface residues and located in the loop region of Cas9 and
each Cas9 portion is capable of fusion to a transcription regulatory domain, wherein said split sites are selected from the split site locations consisting of Cas9 splits at residues 203, 468, 713 and 1153, and when reassembled is operably linked to a plant cell line element.

20. The non-naturally occurring split Cas9 protein system of claim 19 wherein said Cas9 is dCas9 and when reassembled is operably linked to a plant cell line regulatory element.


SEQUENCE LISTING (61 sequences)

SEQ ID NO: 1 - protein, 245 aa, Streptococcus pyogenes
MPKKKRKVGGGSPGGGGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINATSGGGSKRPAATKKAGQAKKKKSR

SEQ ID NO: 2 - protein, 1207 aa, Streptococcus pyogenes
MPKKKRKVGGGSPGGGGSSGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSGGGSKRPAATKKAGQAKKKKSR

SEQ ID NO: 3 - protein, 309 aa, Streptococcus pyogenes
MPKKKRKVGGGSPGGGGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINACLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 4 - protein, 979 aa, Streptococcus pyogenes
MPKKKRKVGGGSPGGGGSMLKKILKIEELDERELIDIEVSGNHLFYANDILTHNSSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSGGGSKRPAATKKAGQAKKKKSR

SEQ ID NO: 5 - protein, 510 aa, Streptococcus pyogenes
MPKKKRKVGGGSPGGGGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKTSGGGSKRPAATKKAGQAKKKKSR

SEQ ID NO: 6 - protein, 942 aa, Streptococcus pyogenes
MPKKKRKVGGGSPGGGGSSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSGGGSKRPAATKKAGQAKKKKSR

SEQ ID NO: 7 - protein, 574 aa, Streptococcus pyogenes
MPKKKRKVGGGSPGGGGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 8 - protein, 979 aa, Streptococcus pyogenes; identical to SEQ ID NO: 4

SEQ ID NO: 9 - protein, 755 aa, Streptococcus pyogenes
MPKKKRKVGGGSPGGGGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVTSGGGSKRPAATKKAGQAKKKKSR

SEQ ID NO: 10 - protein, 697 aa, Streptococcus pyogenes
MPKKKRKVGGGSPGGGGSSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSGGGSKRPAATKKAGQAKKKKSR

SEQ ID NO: 11 - protein, 819 aa, Streptococcus pyogenes
MPKKKRKVGGGSPGGGGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 12 - protein, 734 aa, Streptococcus pyogenes
MPKKKRKVGGGSPGGGGSMLKKILKIEELDERELIDIEVSGNHLFYANDILTHNSSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSGGGSKRPAATKKAGQAKKKKSR

SEQ ID NO: 13 - protein, 1195 aa, Streptococcus pyogenes
MPKKKRKVGGGSPGGGGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKTSGGGSKRPAATKKAGQAKKKKSR

SEQ ID NO: 14 - protein, 257 aa, Streptococcus pyogenes
MPKKKRKVGGGSPGGGGSSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSGGGSKRPAATKKAGQAKKKKSR

SEQ ID NO: 15 - protein, 1259 aa, Streptococcus pyogenes
MPKKKRKVGGGSPGGGGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 16 - protein, 294 aa, Streptococcus pyogenes
MPKKKRKVGGGSPGGGGSMLKKILKIEELDERELIDIEVSGNHLFYANDILTHNSSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSGGGSKRPAATKKAGQAKKKKSR

SEQ ID NO: 17 - protein, 245 aa, Streptococcus pyogenes
MPKKKRKVGGGSPGGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINATSGGGSKRPAATKKAGQAKKKKSR

SEQ ID NO: 18 - protein, 1757 aa, Streptococcus pyogenes
MPKKKRKVGGGSPGGGGSMLKKILKIEELDERELIDIEVSGNHLFYANDILTHNSSGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSILTQVCFNSRLDN

SEQ ID NO: 19 - protein, 309 aa, Streptococcus pyogenes
MPKKKRKVGGGSPGGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINACLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 20 - protein, 1492 aa, Streptococcus pyogenes
MPKKKRKVGGGSPGGGGSMLKKILKIEELDERELIDIEVSGNHLFYANDILTHNSSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSILTQVCFNSRLDN

SEQ ID NO: 21 - protein, 510 aa, Streptococcus pyogenes
MPKKKRKVGGGSPGGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKTSGGGSKRPAATKKAGQAKKKKSR

SEQ ID NO: 22 - protein, 1455 aa, Streptococcus pyogenes
MPKKKRKVGGGSPGGGGSSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSILTQVCFNSRLDN

SEQ ID NO: 23 - protein, 574 aa, Streptococcus pyogenes
MPKKKRKVGGGSPGGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 24 - protein, 1492 aa, Streptococcus pyogenes; identical to SEQ ID NO: 20

SEQ ID NO: 25 - protein, 755 aa, Streptococcus pyogenes
MPKKKRKVGGGSPGGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVTSGGGSKRPAATKKAGQAKKKKSR

SEQ ID NO: 26 - protein, 1210 aa, Streptococcus pyogenes
MPKKKRKVGGGSPGGGGSSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSILTQVCFNSRLDN

SEQ ID NO: 27 - protein, 819 aa, Streptococcus pyogenes
MPKKKRKVGGGSPGGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 28 - protein, 1247 aa, Streptococcus pyogenes
MPKKKRKVGGGSPGGGGSMLKKILKIEELDERELIDIEVSGNHLFYANDILTHNSSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSILTQVCFNSRLDN

SEQ ID NO: 29 - protein, 1247 aa, Streptococcus pyogenes
MPKKKRKVGGGSPGGGGSMLKKILKIEELDERELIDIEVSGNHLFYANDILTHNSSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKEYRSTKEVLDATLIHQSITGLYETRIDLSQLGGDTSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSILTQVCFNSRLDN

SEQ ID NO: 30 - protein, 1195 aa, Streptococcus pyogenes
MPKKKRKVGGGSPGGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKTSGGGSKRPAATKKAGQAKKKKSR

SEQ ID NO: 31 - protein, 770 aa, Streptococcus pyogenes
MPKKKRKVGGGSPGGGGSSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSILTQVCFNSRLDN

SEQ ID NO: 32 - protein, 1259 aa, Streptococcus pyogenes
MPKKKRKVGGGSPGGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 33 - protein, 807 aa, Streptococcus pyogenes
MPKKKRKVGGGSPGGGGSMLKKILKIEELDERELIDIEVSGNHLFYANDILTHNSSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSILTQVCFNSRLDN

SEQ ID NO: 34 - protein, 1259 aa, Streptococcus pyogenes
MPKKKRKVGGGSPGGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFESPTVAYSVLVVAKVEKGKCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 35 - protein, 807 aa, Streptococcus pyogenes
MPKKKRKVGGGSPGGGGSMLKKILKIEELDERELIDIEVSGNHLFYANDILTHNSSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDTSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSILTQVCFNSRLDN

SEQ ID NO: 36 - protein, 556 aa, Streptococcus pyogenes
MPKKKRKVGGGSPGGGGSMLKKILKIEELDERELIDIEVSGNHLFYANDILTHNSSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSRKVDGIGSGSNGSSGSGAIIHAAATSGPEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGKGPGSGATNFSLLETR

SEQ ID NO: 37 - protein, 507 aa, Streptococcus pyogenes
MPKKKRKVGGGSPGGGGSMGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSGSKGEELFTGVVPGGGSEEYKLILNGKTLKGETTTEAVDAATAEKVFKQYANDNGVDGEWTYDDATKTFTVTEGGGSGGGSSRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSGGGSKRPAATKKAGQAKKKKSR

SEQ ID NO: 38 - protein, 185 aa, Streptococcus pyogenes
MPKKKRKVGGGSPGMASRILWHEMWHEGLEEASRLYFGERNVKGMFEVLEPLHAMMERGPQTLKETSFNQAYGRDLMEAQEWCRKYMKSGNVKDLTQAWDLYYHVFRRISKTSRADPKKKRKVEASGSGRADALDDFDLDMLGSDALDDFDLDMLGSDALDDFDLDMLGSDALDDFDLDMLINSR

SEQ ID NO: 39 - protein, 441 aa, Streptococcus pyogenes
MPKKKRKVGGGSPGGGGSMGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSGSKGEELFTGVVPGGGSEEYKLILNGKTLKGETTTEAVDAATAEKVFKQYANDNGVDGEWTYDDATKTFTVTEGGGSADPKKKRKVEASGSGRADALDDFDLDMLGSDALDDFDLDMLGSDALDDFDLDMLGSDALDDFDLDMLINSR

SEQ ID NO: 40 - protein, 546 aa, Streptococcus pyogenes
MPKKKRKVGGGSPGGGGSSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 41 - protein, 307 aa, Streptococcus pyogenes
MPKKKRKVGGGSPGGGGSSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSRADPKKKRKVEASGSGRADALDDFDLDMLGSDALDDFDLDMLGSDALDDFDLDMLGSDALDDFDLDMLINSR

SEQ ID NO: 42 - protein, 834 aa, Streptococcus pyogenes
MPKKKRKVGGGSPGGGGSMLKKILKIEELDERELIDIEVSGNHLFYANDILTHNSSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDTSGGGSKRPAATKKAGQAKKKKSRMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVLES

SEQ ID NO: 43 - protein, 1088 aa, Artificial Sequence
  Engineered sequence based upon rational design
EGRGSLLTCGDVEENPGPTGLSTIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPDPSPKKKRKVEASGGGSKLTS

SEQ ID NO: 44 - protein, 1228 aa, Artificial Sequence
  Engineered sequence based upon rational design
EGRGSLLTCGDVEENPGPTGLSTIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPDPSPKKKRKVEASGGGSKLGGGSTS

SEQ ID NO: 45 - DNA, 1892 bp, Artificial Sequence
  Engineered sequence based upon rational design
ctcttcctcccacagggcctcgagacaattgcgccggcgtctagaggccggccactagtatcaactttgtatagaaaagttggctccgaatttctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagatttcgagctcggtacccggggatcctctagtcagctgacgcgtgctagtggcgcgccgaattcgacccaagtttgtacaaaaaagcaggctgattaccggtcgccaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcactaagcggccgcctcagatctcgagctcaagcttcgaattctgcagtcgacggtaccgcgggcccgggatccaccggatctagataactgatctacccagctttcttgtacaaagtggtacgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttg

SEQ ID NO: 46 - DNA, 53 bp, Artificial Sequence
  Engineered sequence based upon rational design
ggcactctgatttgacaattattcagactaattgtcaaatcagagtgcttttt

SEQ ID NO: 47 - DNA, 53 bp, Artificial Sequence
  Engineered sequence based upon rational design
ggcttgaagtctttaattaaattcagactttaattaaagacttcaagcggttt

SEQ ID NO: 48 - DNA, 1601 bp, Artificial Sequence
  Engineered sequence based upon rational design
ttacagggacagcagagatccagtttggttaattaacaactttgtatagaaaagttggctccgaatttctcgagagcgtagattgtgaacccagtgaaagcgaacgtatgtcgatagattgtgaacccagtgaaagcgaaacgagtccatgtcgatagattgtgaacccagtgaaagcgaaacgcagaccatgtcgatagattgtgaacccagtgaaagcgaaacgttgaccatgtcgatagattgtgaacccagtgaaagcgaatatgacctgtcgatagattgtgaacccagtgaaagcgcgtcaagagtcgatagattgtgaacccagtgaaagcgaaaacgtatgtcgaggtaggcgtgtacggtgggaggcctatataagcagagctcgtttagtgaaccgtcagatcgccctgactagttaagggcccggcgcgcctaaggtacccccgggtaactgatcataattcgacccaagtttgtacaaaaaagcaggctgattaccggtcgccaccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagatgagcggccgcctcagatctcgagctcaagcttcgaattctgcagtcgacggtaccgcgggcccgggatccaccggatctagataactgatctacccagctttcttgtacaaagtggtgaattccggcaattcgatatcaagcttcgaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtggcacctttaagcttcg

SEQ ID NO: 49 - DNA, 1250 bp, Artificial Sequence
  Engineered sequence based upon rational design
atttctcgagagcgtagattgtgaacccagtgaaagcgaacgtatgtcgatagattgtgaacccagtgaaagcgaaacgagtccatgtcgatagattgtgaacccagtgaaagcgaaacgcagaccatgtcgatagattgtgaacccagtgaaagcgaaacgttgaccatgtcgatagattgtgaacccagtgaaagcgaatatgacctgtcgatagattgtgaacccagtgaaagcgcgtcaagagtcgatagattgtgaacccagtgaaagcgaaaacgtatgtcgaggtaggcgtgtacggtgggaggcctatataagcagagctcgtttagtgaaccgtcagatcgcctagattgtgaacccagtgaatggaaaattggtcttgacaaaaagaactagattgtgaacccagtgaatggattgtctgttgtgcccagttagattgtgaacccagtgaatggactagtccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagcggccgcctcagatctcgagctcaagcttcgaattctgcagtcgacggtaccgc

SEQ ID NO: 50 - DNA, 92 bp, Artificial Sequence
  Engineered sequence based upon rational design
ctatctgcactagatgcaccttactatctgcactagatgcaccttactatctgcactagatgcaccttactatctgcactagatgcacctta

SEQ ID NO: 51 - DNA, 88 bp, Artificial Sequence
  Engineered sequence based upon rational design
tcaacatcagtctgataagctatcaacatcagtctgataagctatcaacatcagtctgataagctatcaacatcagtctgataagcta

SEQ ID NO: 52 - DNA, 92 bp, Artificial Sequence
  Engineered sequence based upon rational design
cagctgcttttgggattccgttgcagctgcttttgggattccgttgcagctgcttttgggattccgttgcagctgcttttgggattccgttg

SEQ ID NO: 53 - DNA, 94 bp, Artificial Sequence
  Engineered sequence based upon rational design
ccgcttgaagtctttaattaaaccgcttgaagtctttaattaaaccgcttgaagtctttaattaaacccgggccgcttgaagtctttaattaaa

SEQ ID NO: 54 - DNA, 88 bp, Artificial Sequence
  Engineered sequence based upon rational design
aagcactctgatttgacaattaaagcactctgatttgacaattaaagcactctgatttgacaattaaagcactctgatttgacaatta

SEQ ID NO: 55 - DNA, 92 bp, Artificial Sequence
  Engineered sequence based upon rational design
tgtgcaaatctatgcaaaactgatgtgcaaatccatgcaaaactgatgtgcaaatctatgcaaaactgatgtgcaaatccatgcaaaactga

SEQ ID NO: 56 - DNA, 278 bp, Artificial Sequence
  Artificial sequence based upon rational design
catgtgattacgccaagctacgggcggagtactgtcctccgagcggagtactgtcctccgagcggagtactgtcctccgagcggagtactgtcctccgagcggagttctgtcctccgagcggagactctagatacccctctccgcttctgaattctaggcgtgtacggtgggaggcctatataagcagagctcgtttagtgaaccgtcagatcgcctcgagtacccctctccgcttcttacccctctccgcttcttacccctctccgcttctggatcc

SEQ ID NO: 57 - DNA, 295 bp, Artificial Sequence
  Engineered sequence based upon rational design
tcatgtgattacgccaagctacgggcggagtactgtcctccgagcggagtactgtcctccgagcggagtactgtcctccgagcggagtactgtcctccgagcggagttctgtcctccgagcggagactctagatatagaaccgatcctcccattgaattctaggcgtgtacggtgggaggcctatataagcagagctcgtttagtgaaccgtcagatcgcctcgagtatagaaccgatcctcccatttatagaaccgatcctcccatttatagaaccgatcctcccattggatcc

SEQ ID NO: 58 - DNA, 356 bp, Homo Sapiens
catcgggggagcagtcctccaaggactggccagtctccagatgcccgtgcacacaggaacactgccttatgcacgggagtcccagaagaaggggtagatttctttccccaccttagttacaccatcaagacccagccagggcatcccccctcctggcctgagggccagctccccatcctgaaaaacctgtctgctctccccacccctttgaggctatagggcccaaggggcaggttggactggattcccctccagcccctcccacccccaggacaaaatcagccaccccaggggcagggcctcacttgcctcaggaaccccagcctgccagcacctattccacctcccagcccagc

SEQ ID NO: 59 - DNA, 454 bp, Homo Sapiens
ggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgaccccttccgggtttccggcccagccccttccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 60 - DNA, 20 bp, Artificial Sequence
  Engineered sequence based upon rational design
tacgttctctatcactgata

SEQ ID NO: 61 - DNA, 20 bp, Artificial Sequence
  Engineered sequence based upon rational design
tagattgtgaacccagtgaa